(19) United States
(12) Patent Application Publication
(10) Pub. No.: US 2003/0099693 A1
(43) Pub. Date: May 29, 2003

(54) Wafer

(76) Inventors: Anthony David Auffret, Sandwich (GB); Gillian Margaret Eccleston, Sandwich (GB); Michael John Humphrey, Sandwich (GB); Kerr Hugh Matthews, Sandwich (GB); Howard Norman Ernest Stevens, Sandwich (GB)

Correspondence Address: PFIZER INC., PATENT DEPARTMENT, MS8260-1611, EASTERN POINT ROAD, GROTON, CT, 06340, US

(21) Appl. No.: 10/285,072

(22) Filed: Oct. 30, 2002

Related U.S. Application Data
(60) Provisional application No. 60/340,973, filed on Dec. 7, 2001.

(30) Foreign Application Priority Data
Nov. 2, 2001 (GB) 0126389.6

(51) Int. Cl. A61K 31/47; A61K 31/4545; A61K 9/70

(52) U.S. Cl. 424/443; 514/307; 514/326; 514/318

(57) ABSTRACT

The present invention concerns a wafer composition comprising: a polymer substrate; a surfactant; and water; characterised in that the wafer further comprises stable, i.e. stable of size and form, crystalline particles of a pharmaceutically active wound healing agent.

The present invention relates to a wafer composition, in particular, a lyophilised wafer which comprises stable crystalline particles of a pharmaceutically active agent, particularly a wound healing agent.

Conventional wound dressings take the form of absorbent pads of fibrous material such as lint or non-fibrous materials such as dehydrated hydrogels. The wound dressing is intended to form a barrier to external contaminants whilst allowing air to permeate the wound and allowing excess moisture to be drawn from the wound. Known wound dressings may also incorporate pharmaceutically active compounds to promote wound healing.

Generally, pharmaceutical actives when directly applied to the surface of a wound are applied in the form of solutions, powders, sprays, ointments, creams or gels. These modes of delivery are advantageous in ensuring delivery of the medicament to the tender area of an open or closed wound. However, one problem with such directly applied formulations is that it is difficult to provide a known amount of the active substance to the wound. Furthermore, the active substance is not always applied evenly to the area of the wound and local concentrations, thus, vary across the wound. There is the additional disadvantage that such modes of application do not lend themselves to the controlled release of the pharmaceutically active ingredient.

The prior art contains a number of references to lyophilised vehicles which may enable the delivery of an active ingredient to a wound.

International Patent Application Publication Number (WO) 97/39781 discloses a method of producing a dehydrated hydrogel comprising dispersing fibres into an aqueous solution of a hydrogel precursor material incorporating a plasticiser, the fibres incorporating cations which are capable of cross-linking said precursor material to form a hydrogel, and evaporating water to produce a dehydrated hydrogel. Thus, fibres and a plasticiser are essential features of the disclosure in order to produce a stable dehydrated hydrogel. The hydrogel may incorporate an active ingredient for delivery to a wound, but no details are given of the physical properties of the active material, particularly the use of crystalline particles with a surfactant to aid dispersion.

WO95/00184 discloses a dried hydrogel that can be cut into shape for wound healing, where freeze-drying may be employed and where further active agents can be employed. Once again, there is no mention of the use of crystalline particles with a surfactant to aid dispersion.

WO95/18635 discloses a pharmaceutical composition for treating a wound comprising a matrix of hyaluronic acid containing at least one peptide or protein with wound healing activity. The matrix is formed by freeze-drying an aqueous solution of the peptide or protein and hyaluronic acid. Again there is no mention of the use of crystalline particles with a surfactant to aid dispersion.

WO99/01166 discloses a method for preparing a non-fibrous porous material being swellable but not soluble in water, said material essentially consisting of one or more polymers and one or more pharmaceutical agents, by dehydrating an aqueous solution or gel of the components and subjecting the resulting porous material to dry heat treatment. The pharmaceutically active agents include protease inhibitors and metalloprotease inhibitors. Dry heat treatment used in the production of the material causes cross-linking to occur so that the material does not disintegrate in water. There is no mention of the use of crystalline particles with a surfactant to aid dispersion and furthermore, a dry heat process is employed to stabilise the matrix.

U.S. Pat. No. 6,117,437 discloses a sheet-like wound dressing containing an active substance in homogeneously dispersed form. The material is prepared by coating the material onto a flat substrate and cooling or drying. The examples disclose the use of organic solvents which are dried in drying channels at elevated temperature. The examples do not describe the formation of wafers, only thin films, and there is no mention of the particular use of crystalline particles with a surfactant to aid dispersion.

German patent application no. (DE) 4328329 discloses a biomatrix formed from polysaccharides and incorporating pharmaceutically active agents for application to a wound. The biomatrix may optionally include fibres. There is no mention of the use of crystalline active agents in the matrix. Furthermore, the applicants have found that the freeze-drying techniques employed in DE4328329 are completely unsuitable for forming biomatrices which do not comprise viscose fibres. By following the experimental conditions set out in example 3 of DE4328329, the applicants found that the frozen solution melted during the high temperature freeze-drying process. Only when viscous fibres, an optional component of DE4328329, are employed in the composition could collapse of the biomatrix be avoided at such high temperatures. Even then, an open network structure for the biomatrix is unlikely to be maintained.

WO00/36353 discloses freeze-dried polymer gel formulations which may comprise active agents and which may be applied to wounds. There is no specific disclosure of the physical characteristics of the active ingredients, i.e. whether crystalline materials may be employed.

More recently, WO02/34304, published after the priority date of the present invention, discloses self-adhesive hydratable matrices for topical therapeutic use. Once again, there is no specific disclosure of the physical characteristics of any active ingredients, i.e. whether crystalline materials may be employed.

Thus, the prior art alludes to the use of wound healing agents incorporated within freeze-dried matrices, but is silent as to the use of insoluble and crystalline forms of such agents dispersed in an aqueous polymeric structure. Thus, there is no commentary on the likely problems associated with incorporation of such crystals within a wafer. Freeze dried matrices incorporating active ingredients suitable as wound healing pads or bandages need to have some degree of flexibility to make full contact with a wound. The flexibility of a disperse freeze-dried material, such as a wafer, can be consider to have two components. Firstly, that arising from mechanical displacement of the individual strands of the wafer. If those strands are vitrified, as is the case in most freeze-dried products, then they will have a brittle nature. Thus, the inherent plasticity of the strand forming material will contribute to the overall flexibility of the freeze-dried material. This second contribution to the flexibility is influenced by the water in the material, which will act as a plasticiser; the more water in the system, the more flexible the dried matrix will be. It is usual to state the water content as a mass fraction of the total composition e.g. % w/w or weight of water/weight of composition. If that composition contains a phase separated crystalline material, that is not a hydrated crystal, then the reported water content will underestimate the actual water content of the non-crystalline or amorphous matrix. It is well-known that crystalline materials undergo crystal growth in an aqueous environment due to the effect of Ostwald Ripening. Such an effect can lead to non-uniformity of the wound healing product and its unsuitability in providing a pre-determined amount of active material to the wound site.

The present applicants have been involved in the development of wound healing treatments comprising crystalline pharmaceutically active wound healing agents. Initial investigations on drug delivery centred on use of gels incorporating the active materials under development. However, problems were encountered in the storage stability of the active ingredient within the gel formulations, particularly following sterilisation, owing to crystal growth and polymorphic transition. One solution is to freeze-dry the polymer to remove water, but the efficacy of the result will depend upon the final water content and can be fraught with problems. It is possible by freeze-drying to reduce the water content of a composition to less than 2% w/w. In order to maintain a product at such a low water content, typically the product must be packaged with an effective barrier against water vapour ingress from the atmosphere. In the absence of such a barrier, the present applicants found that the placebo matrices had a high water content, as much as 20%, which is thought to act as a plasticiser contributing to the flexibility of the material. For drug-loaded wafers, it was considered that crystal growth and possibly the polymorphic transitions previously seen would be likely to occur at such high water contents. However, it has now been surprisingly found that, at high drug contents, greater than 30 mg/ml, low water contents of less than 5% prevail, even in the absence of an effective barrier. Conversely, at low drug contents, less than 30 mg/ml, higher water contents of 10-20% prevail in the absence of an effective barrier. In both cases there is no need for a specialised barrier packaging and thus a physically stable wound healing composition can be produced in which the crystal form is stabilised from polymorphic transition.

Thus, the present invention provides a wafer composition comprising:

(a) a polymer substrate;

(b) a surfactant; and

(c) water;

characterised in that the wafer further comprises stable, i.e. stable of size and form, crystalline particles of a pharmaceutically active wound healing agent.

According to the present invention, a wafer may be defined as a light and highly porous structure. The wafer compositions of the present invention are stable, without requiring stabilising fibres, and provide a means for administering wound healing agents to wounds in a controlled and consistent manner. Preferably, the wafers are lyophilised (freeze-dried).

As a further preferred aspect of the invention, the wound healing agents comprise uPA or MMP-3 and/or -13 inhibitors. Suitable wound healing agents include those particularly described in EP0568289, WO9811089, WO9905096, WO9940088, U.S. Pat. No. 5,849,866, WO9005719, WO9627583, WO9907675, WO9833768, WO9935124, WO9929667, WO0074681, WO9920608 and WO0005214, the compounds disclosed in which are incorporated herein by reference. Most preferably, the wound healing agent is selected from the group consisting of 3-(4-Chloro-1-guanidino-7-isoquinolyl)benzoic acid), 2-[(4-Chloro-1-guanidino-7-isoquinolinyl)sulfonylamino]isobutyric acid), (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid) (hereinafter described as compound A), N-Hydroxy-4-[(4-{4-[6-(2-hydroxyethoxy)-2-pyridyl]-3-methylphenyl}-1-piperidinyl)sulfonyl]tetrahydro-2H-pyran-4-carboxamide) or a combination thereof. The wound healing agent content in the pre-formed mixture is suitably in the range 0.1 to 300 mg/ml of mixture, more suitably 0.1 to 100 mg/ml, preferably in the range 1-100 mg/ml, most preferably 10-60 mg/ml. The exact concentration of active ingredient to be used in the wafer depends on the activity and physical properties of the wound healing agent, the type of wound to be treated and the patient to be treated.

Further suitable wound healing agents include other protease inhibitors, such as neutral endopeptidase inhibitors, enzyme inhibitors such as those of phosphodiesterase, nuclear hormone receptor agonists such as estrogens, growth factors and cell surface receptor modulators such as GPC receptor agonists, for example A2a agonists or antagonists and compounds active at ion channels.

The wafer compositions of the present invention are applied directly to open wounds and, hence, care has to be taken in applying correct efficacious doses, without achieving excessive concentrations that could lead to adverse effects. This point can be illustrated with the use of selective protease inhibitors, which have been described for uPA and MMP-3 and/or -13. These enzymes have been shown to be up-regulated in chronic ulcers, and there is evidence implicating these enzymes in the degradation of cellular matrix and the pathology of ulceration. However, normal healing wounds require some regulated proteolysis to allow matrix remodelling and cell migration to occur. Hence it is essential that pro-healing proteases like plasmin, MMP-1, MMP-2, MMP-9, MMP-14, and tPA are not inhibited.

Hence appropriate formulation design is required, whereby the solubility of the candidate in the formulation and wound fluid is controlled to ensure that excessive concentrations and loss of selectivity is prevented. The solubility range to achieve efficacious doses without adverse effects for a range of protease inhibitors is shown in Table 1.

TABLE 1

| | Concentration* for 50% enzyme inhibition (mg/ml) | | | |
|---|---|---|---|---|
| MMP-3 | MMP-3 | MMP-2 | MMP-9 | MMP-14 |
| Compound | 0.01 | 15.6 | 9.5 | 20.4 |
| A | | | | |
| UPA | uPA | TPA | Plasmin | — |
| inhibitor X | 0.004 | >11.3 | 11.3 | — |

*Adjusted for Plasma Protein Binding

Inhibitor X in Table 1 refers to 3-(4-Chloro-1-guanidino-7-isoquinolyl)benzoic acid).

Thus, as a further aspect of the present invention, the wound healing agents preferably have low water solubility, suitably in the range 1 ng/ml to 10 mg/ml, preferably in the range 4 ng/ml to 5 mg/ml.

As a further aspect, combinations of two or more crystalline wound healing agents are envisioned by the present invention. Furthermore, the crystalline wound healing agent(s) may be combined with one or more other agents, which may be crystalline, amorphous or soluble in the pre-formed wafer mixture, e.g. a cytokine such as a growth hormone or a polypeptide growth factor, bacteriostatic or bacteriocidal compounds, e.g. iodine, iodopovidone complexes, chloramine, chlorohexidine, silver salts such as sulphadiazine, silver nitrate, silver acetate, silver lactate, silver sulphate or silver chloride, zinc or salts thereof, metronidazole, sulpha drugs and penicilins, tissue-healing enhancing agents, e.g. RGD tripeptides and the like, proteins, amino acids such as taurine, vitamins such as ascorbic acid, enzymes for cleansing of wounds, e.g. pepsin, trypsin and the like, cytotoxic agents and proliferation inhibitors for use in, for example, surgical insertion of the product in cancer tissue and/or other therapeutic agents which may be used for topical application, pain relieving agents such as lidocaine or chichocaine, emollients, retinoids, or agents having a cooling effect.

It will be apparent to the person skilled in the art that other classes of crystalline therapeutic agents may be incorporated into wafers according to the present invention, in place of wound healing agents. Thus, as an alternative aspect of the present invention, there is provided a wafer comprising a polymer substrate; a surfactant; and water; characterised in that the wafer further comprises stable, i.e. stable of size and form, crystalline particles of a pharmaceutically active agent. Wafers incorporating such therapeutic agents may be useful for administration of active ingredient through any wound surface absorbing mucosal, or similar, surface. Suitable surfaces are present in oral, nasal, rectal and vaginal mucosae and on the surface of the eye. Suitable therapeutic agents envisioned by the present invention are, thus, those which may be administered to the afore-mentioned surfaces to treat disorders related to such mucosal surfaces. Alternatively, the mucosal surface may merely provide a suitable means of administering and absorbing a therapeutic agent for any disorder treatable in such a manner.

Any suitable rapidly rehydratable polymer may be used in the wafer of the present invention. The polymer may be formed by a chemical reaction in situ in the aqueous solution from suitable monomers or a pre-formed water-soluble polymer may be dissolved in water to form the hydrogel. Suitable polymers according to the present invention may be used alone or in combination and include: Natural Polysaccharides such as acacia (gum arabic), pullulan, inulin, agar, alginic acid and its salts, kappa-/iota carrageenan, chitosan, gellan gum, glycyrrhizin, guar gum, hyaluronic acid/sodium hyaluronate, hyaluronic acid esters, karaya gum, locust bean gum (carob gum), pectin starch and derivatives, tragacanth gum and xanthan gum; Semisynthetic Polysaccharides such as propylene glycol alginate, CMC and CMC sodium, HEC, HEMC, HPC, HPMC and MC; Synthetic Polymers such as Carbomer (different Carbopol®grades), poloxamer (Pluronic® grades), polyacrylamide glyceryl polyacrylate, PEGs, PVA and povidone; Colloidally Dispersed Solids such as microcrystalline silica, microcrystalline cellulose, microcrystalline cellulose+CMC sodium (Avicel RC-591) and clays (e.g. Bentonite) and proteins such as collagen and gelatin. Particularly preferred is xanthan gum. A suitable concentration of polymer in pre-lyophilised mixture depends on the viscosity of the polymer. For example, for xanthan, a suitable aqueous concentration is in the range 0.5 to 1% w/v of aqueous solution (5 to 10 mg/ml) with 1-30 mg/ml of active ingredient. For pullulan, which has a lower viscosity than xanthan, a suitable concentration is in the range of about 3 to 10% w/v (30 to 100 mg/ml) with 1-30 mg/ml of active ingredient.

The purpose of the surfactant is to provide a homogenous mixture of wafer components prior to processing. Suitable surfactants according to the present invention include Tween 20®, Tween 80®, Lutrol F-68® and Lutrol F-127®, most suitably Lutrol F-68 and Lutrol F-127. It has also been found that, for (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid), an alkaline solution of the pre-formed wafer mixture possesses the desired properties of a surfactant and is, thus, included within the definition of surfactant according to the present invention. For the avoidance of doubt, the polymer substrate and the surfactant may comprise the same material. A typical concentration for surfactant is in the range 0.02-5% w/w based on the pre-formed (pre-lyophilised) formulation, more suitably 0.2%.

The water content of the wafer according to the present invention is suitable to provide sufficient flexibility to the matrix. Typically, the water content is in the region of 2-20% w/w, suitably 2-15% w/w, alternatively 5-20% w/w or 13-17% w/w, based on the composition of the wafer.

The preferred method of forming the wafer is by lyophilisation (freeze-drying). A wafer according to the present invention may be prepared by forming a suspension by mixing a polymer substrate, a surfactant and a wound healing agent in water, shaping the suspension into a suitable mould and lyophilising the shaped composition. The resulting wafer may be optionally sterilised using suitable techniques such as gamma irradiation or treatment with ethylene oxide.

Freeze-drying or lyophilisation of polymers can produce a shaped material of a highly porous nature. The principle of freeze-drying is well known. An aqueous solution or mixture of a soluble polymer, a surfactant and a wound healing agent is frozen. Crystals of ice form between polymer molecules, which may be removed by sublimation on application of a vacuum. The remaining polymer forms a matrix with the residual water.

The freeze-drying process of the present invention comprises a two step process. In the initial stage, which is referred to as primary freeze-drying, water removal occurs by the sublimation of ice. In the second stage, referred to as secondary freeze-drying, control of the final water content allows fine-tuning of the mechanical properties of the wafer.

During the primary freeze-drying step, the aqueous solution of polymer is cooled from room temperature to below 0° C. wherein the water molecules begin to nucleate and freeze as ice crystals. Ice crystals grow throughout the solution leading to a concentration of the residual solution. More ice forms as the temperature is lowered and consequently the residual solution becomes more concentrated. This freeze-concentrate occupies the interstitial spaces between the ice crystals and is spread throughout the volume of the original solution. If cooled sufficiently it will vitrify, forming a chemically and mechanically stable glass. The shaped biomatrix is formed in this freezing step. The bulk of the water separation occurs in this step and the physical conditions are arranged such that a vacuum is applied with a calculated amount of heating to ensure that there is a heat-mass transfer. Thus, sufficient energy is applied to sublime the ice whilst at the same time keeping the temperature below the glass transition temperature.

In the secondary drying step, a portion of the water remaining in the unfrozen interstitial freeze-concentrate is removed. During this stage of the process the controlled application of heat, under vacuum, effectively results in the removal of this portion of the residual water by desorption to the gas phase. The low water content of the wafer of the present invention is an important factor in its stability and strength. It is therefore possible in the secondary freeze-drying step to reduce the water content of the biomatrix in the range of 5-20% by weight, suitably in the region of 13-17%. Alternatively, the water content may be reduced below these values in the drying process, down to the region of 2% w/w, then the water allowed to equilibrate to the suitable and preferred values above by exposure to an atmosphere of an appropriate relative humidity.

Freeze-drying is not the only means of producing a wafer according to the present invention. Electrohydrodynamic (EHD) spraying may also be used, a technique described, for example, in WO01/27365, the methods described in which are incorporated herein by reference. Furthermore, wafers may be produced by any convenient spinning process that produces fine filaments which can be laid down in a mould or otherwise constrained into a wafer form. Such technologies as might be familiar to those skilled in the art are the process of melt extrusion, which forces mixtures of components, subject to heat and pressure treatment through an orifice of defined and controllable form, and also by the process of melt spinning exemplified in U.S. Pat. No. 6,116,880.

The process of sterilisation of the wafer according to the present invention by means of gamma irradiation involves exposure to gamma rays generated by a radioactive cobalt-60 source in a specially designed irradiation cell. The principle underlying this sterilisation process is that high energy radiation is powerful enough to destroy completely biological contaminants without being sufficiently powerful to damage the material being sterilised.

A semi-solid liquid formulation, having a similar composition to that which might be used to form a freeze-dried wafer may be difficult to sterilise by conventional means. Gamma-irradiation can destroy the structure (viscosity) either during the process or permanently. Loss of viscosity can lead to aggregation of insoluble particles and/or an initiation of crystal maturation with consequent perturbations of particle size distribution. This can cause significant problems in terms of the rate of release of the agent into the wound or its rate of absorption within the wound.

Although a dried dosage form can be affected by gamma-irradiation, this can result in a loss of viscosity of a rehydrated form, compared to the original solution. However, as long as the dosage form remains in the dry state there is no effective change in relevant mechanical properties of the dried form. The wafers of the present invention, particularly those where xanthan gum is the polymer substrate, may be sterilised using gamma irradiation without significantly affecting the particle size distribution of the active ingredient.

The invention may be illustrated by the following non-limiting examples in which:

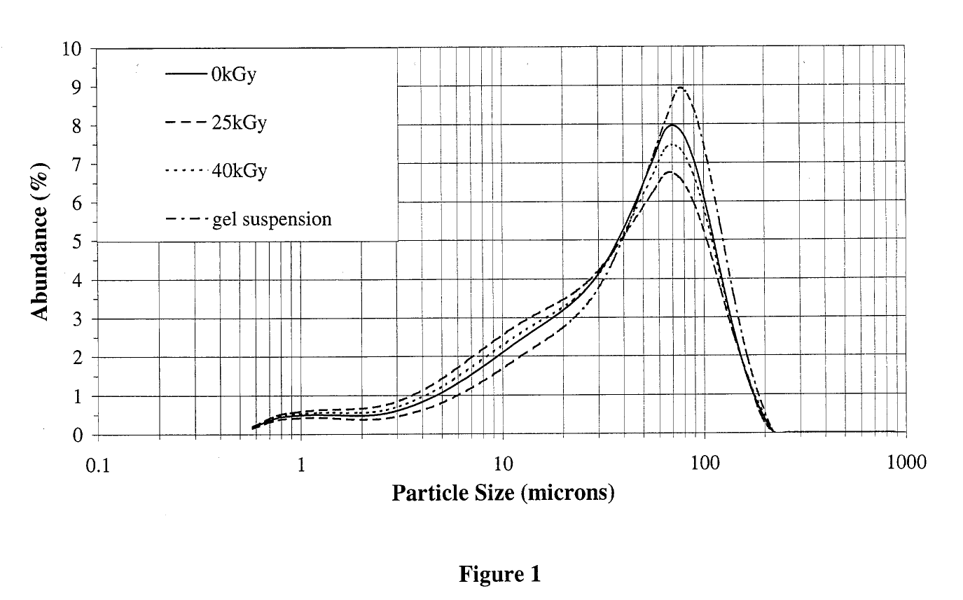

FIG. 1 shows particle size distributions for the suspension and wafers of Example 4 at time zero;

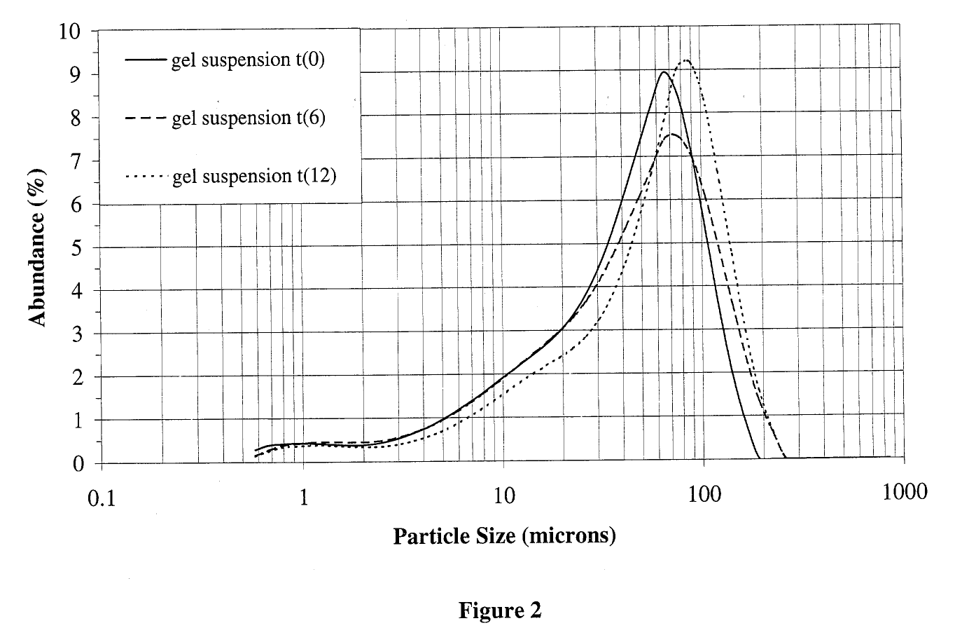

FIG. 2 shows particle size distributions for the gel suspension of Example 4 at 0, 6 and 12 weeks (40° C.);

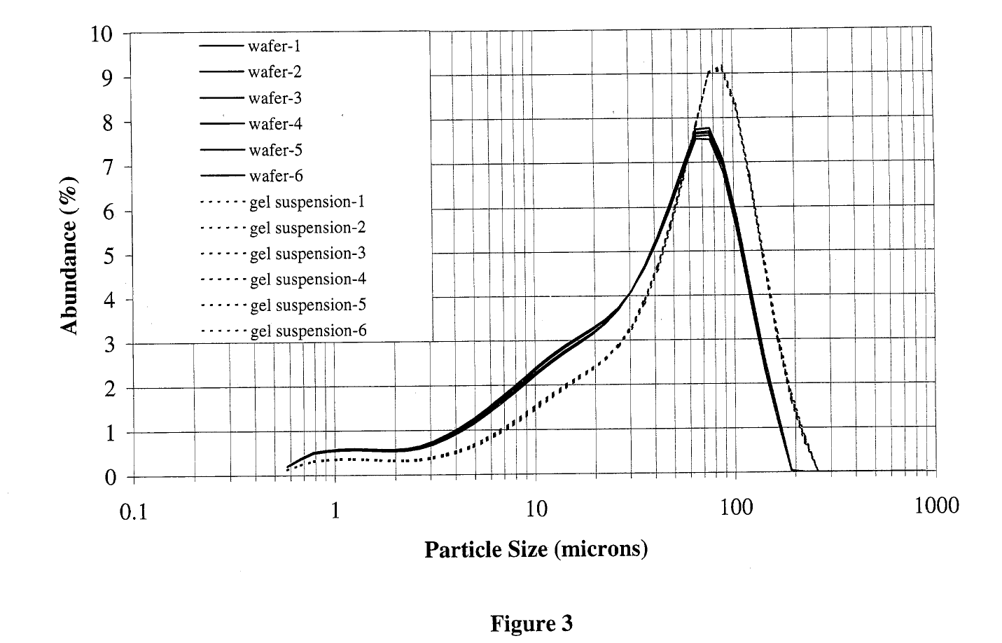

FIG. 3 shows particle size distributions for the gel suspension and non-irradiated wafer of Example 4 at 12 weeks (40° C.); and

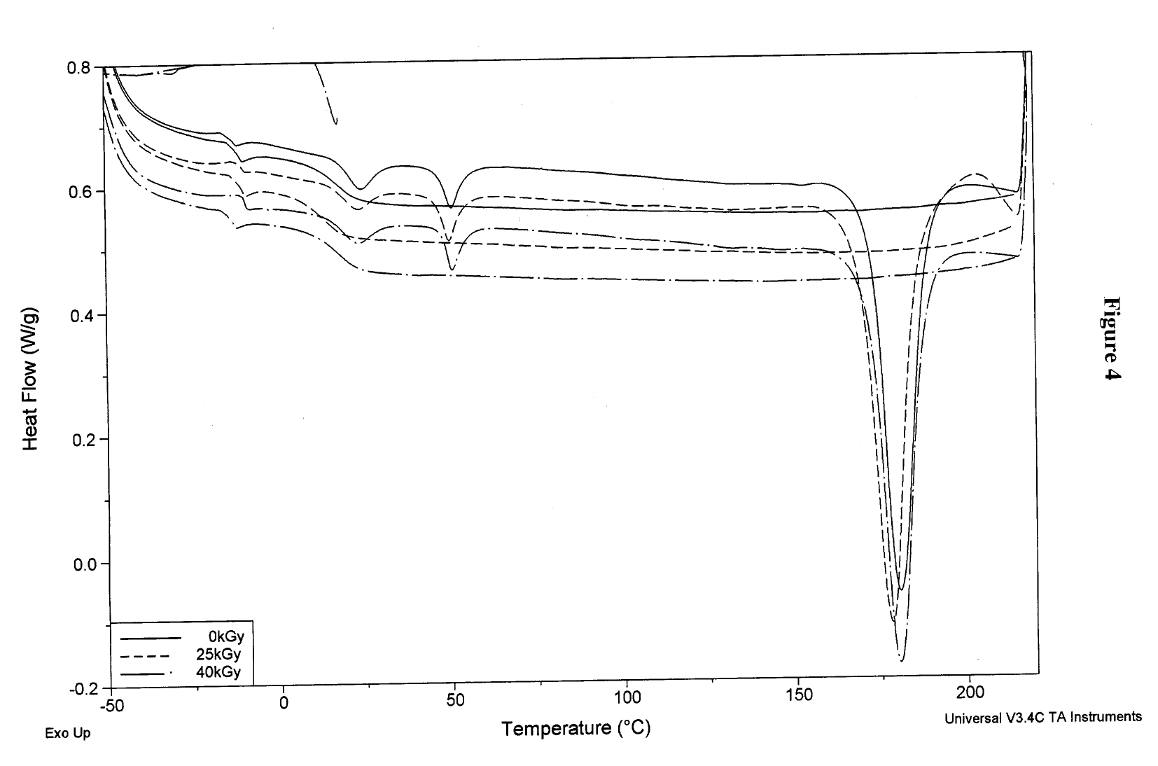

FIG. 4 shows DSC results for the irradiated and non-iradiated wafers of Example 4 stored at 40° C. for 3 weeks (1^{st} and 2^{nd} heat from −50 to 220° C. at 5° C./min).

### GENERAL PREPARATION OF POLYMER SUSPENSION

The wound healing agent, compound A, is suspended in deionised water with the aid of surfactant. Typically, 0.2% w/v Lutrol F-68 is used. Xanthan gum is dissolved in the drug suspension at a concentration of 0.5% w/v and mixed until a uniform suspension is obtained. The mixture is poured in a suitable mould in a quantity commensurate with the desired size and shape of the final product. The drug loading may be varied from 0.1-30.0 mg/ml of the pre-lyophilised mixture, dependent on the desired dose per unit area. The cast mixture of polymer, surfactant and drug is then subjected to one of the following freeze-drying processes.

### EXAMPLE NO. 1

The mixtures, in moulds, are placed in the freeze-drier and frozen to −55° C. in stages over a period of 8 hours. The stages are typically, +22 (room-temperature) to −25° C. in 3 hours; −25 to −35° C. in 2 hours; −35 to −45° C. in 2 hours and −45 to −55° C. in 1 hour. When the mixtures reach the target temperature of −55° C., the freezing stage, a vacuum, P≦SVP (saturation vapour pressure), preferably 30% of SVP, is applied and maintained for the rest of the cycle. The oven temperature is then increased in a single thermal ramp to +10° C. and maintained at this temperature for 4 hours. This is the bulk, or primary, drying phase. The oven temperature is then raised again in 3 thermal ramps to +22° C. (viz +10 to +15° C.; +15 to +20° C. and +20 to +22° C.) over a period of 12 hours. The last 8 hours are considered to be the secondary drying phase. After a total period of 25 hours, the moulds are removed from the freeze-drier and the lyophilised wafers easily recovered from the moulds.

### EXAMPLE NO. 2

The mixtures, in moulds, are placed in the freeze-drier and frozen to −45° C. in a single stage. Ten hours are allowed for the freezing stage. A vacuum, P≦SVP (saturation vaporisation pressure), preferably 30% of SVP, is applied and maintained for the rest of the cycle. The oven temperature is then raised to −30° C. gradually over a period of 32 hours. The primary drying stage is complete 16 hours after vacuum application. The succeeding 16 hours are considered to be part of the secondary drying stage. Subsequently, the oven temperature is increased gradually, over 12 hours, to +20° C. The total time allotted to secondary drying is 28 hours. The total freeze-drying cycle from start to finish is 54 hours. The moulds are removed from the freeze-drier and the lyophilised wafers easily recovered.

### EXAMPLE NO. 3

The mixtures, in moulds, are placed in the freeze-drier and frozen to −25° C. in a single stage. When the target temperature is obtained, a vacuum, P≦SVP, preferably 30% of SVP, is applied and the oven maintained in an isothermal state for 5 hours. The precise time required for primary drying will depend on the total mass of formulation to be lyophilised. Of particular importance is the fill depth and area of the mixture as cast. For example, a disc measuring 3.5 cm in diameter and 0.5 cm thick (V=4.8 cm³) requires 2.5-5.0 hours. A square wafer measuring 10×10 cm and 1 mm thick (V=10 cm³) requires 5.0-10.0 hours depending on the vacuum conditions and the condenser efficiency. The oven temperature is then increased at 5° C./hour to +20° C. enabling secondary drying. Typically, for wound healing wafers, a residual water content of 2-20%, more preferably, 2-15%, requires a secondary drying stage of 12 hours at +20° C. For water contents lower than the specified range, increasing the oven temperature and/or prolonging the drying time is necessary. However, the drying temperature should not exceed the collapse temperature (softening temperature) of the product. For the examples cited, the secondary drying temperature should not exceed 50° C. More preferably, a secondary drying temperature in the range 20-40° C., even more preferably 20-30° C. is used. On completion of the drying process, the moulds are removed from the freeze-drier and the lyophilised wafers easily recovered.

### EXAMPLE NO. 4

4.1 Method

Lutrol F68 (0.12 g) was dissolved in deionised water (57.48 g) contained in a sealable glass vial (100 ml) with manual agitation. (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid) (1.80 g) was added to this solution and the mixture homogenised using a high-shear stirrer (8 mm mixer-head, 24,000 rpm for 5 minutes). XG (0.60 g ,Xantural 180, Kelco) was added and the vial stoppered and crimped with a metal top. This mixture was placed on a rotary mixer for at least twelve hours or until all the suspending agent was completely dissolved and a smooth, viscous suspension obtained. If necessary, the suspension may be degassed, by application of a vacuum, to remove contained air-bubbles resulting from high-shear stirring.

The bulk of the suspension was cast to 24-well polystyrene microplates (moulds) and freeze-drying undertaken according to Example No.3 of the present application. On completion of the freeze-drying process, wafers were removed from their moulds and divided into three equal lots, one of which was kept for reference.

4.2 Gamma-Irradiation of Wafers

Two thirds of the wafers produced were gamma-irradiated, one-third at 25 kiloGrays (kGy) and the rest at 40 kGy. After irradiation, all samples, including the non-irradiated reference samples, were regrouped and split into two equal lots. One half was placed in a stability cabinet set to 25° C. and the other half placed in a stability cabinet set at 40° C. For comparative purposes, samples of drug suspensions were retained in sealed glass vials and stored with the wafers.

4.3 Determination of Particle Size

Determination of particle size distributions in both gel suspensions and wafers was undertaken in a small sample dispersion cell located within a Malvern Mastersizer S laser light scattering instrument. The cell was filled with a dilute solution of Lutrol F68 in deionised water (0.2% w/v) to aid sample dispersion and individual gel suspensions administered directly using disposable micropipettes. It should be noted that wafer samples were rehydrated to the gel form by addition of deionised water (2 ml) before analysis. Scattered light was detected at the rate of 25,000 measurements per second for 10 seconds and the results displayed as particle size distributions, “Abundance” (%) vs. Particle Size (microns). Calculation of the mean particle size, or apparent volume D[4,3], was computed by the instrument software and based on the statistics of polydisperse systems.

4.4 Thermal Analysis of Drug-Loaded Wafers

Thermal analysis by Differential Scanning Calorimetry (DSC) was conducted on three drug-loaded wafers, stored at 40° C. for three weeks, to assess the presence and form of both Lutrol F68 and (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid). Two of the samples selected for analysis had been gamma-irradiated at 25 and 40 kGy respectively while the remaining sample was of a non-irradiated wafer. All drug-loaded wafers were contained in hermetically sealed aluminium pans to prevent the evaporation and loss of residual water at elevated temperatures during measurement. Such a precaution stabilises the heat capacity and improves baseline stability. Samples were initially cooled to −50° C. and following a short period of equilibration, heated to 220° C. at 5° C./min. Subsequent cooling at a controlled rate (5° C./min) to −50° C. was followed by a repeat of the heating cycle. The results are displayed in FIG. 4.

4.5 Results & Discussion

Mean particle sizes and the standard deviation for all analysed samples are summarised in Table 2.

TABLE 2
Mean particle sizes measured for irradiated/non-irradiated wafers
and the gel suspension from which they were made.

| | Mean Particle Size & Standard | | | | | | | | |
|---|---|---|---|---|---|---|---|---|---|
| | Deviation (microns) | | | | | | | | |
| | | | | | | | Gel | | |
| | 25° C. store | | | 40° C. store | | | | suspension | |
| Time | Irradiation dose (kGy) | | | | | | | Time | |
| (weeks) | 0 | 25 | 40 | 0 | 25 | 40 | | (wks) | Mean |
| 0 | 47.5 | 43.9 | 46.5 | — | — | — | Mean | 0 | 47.5 |
| | 0.7 | 0.3 | 0.4 | — | — | — | δ | | 0.7 |
| 3 | 46.7 | 47.3 | 48.1 | — | 47.4 | 47.2 | | 3 | 52.5 |
| | 0.7 | 0.4 | 0.9 | — | 0.8 | 1.2 | | | 1.7 |
| 6 | 48.8 | — | — | 48.6 | 46.4 | 46.2 | | 6 | 59.2 |
| | 0.9 | — | — | 0.9 | 0.9 | 1.3 | | | 4.4 |
| 12 | — | — | — | 48.0 | 48.3 | 46.0 | | 12 | 62.0 |
| | — | — | — | 0.8 | 0.4 | 0.7 | | | 0.5 |

Data for samples stored at 25° C. for a maximum period of six weeks produce a range of values from 43.9 to 48.8 μm with an average deviation of 0.6 μm irrespective of whether they are irradiated or not. Accelerated ageing at 40° C. produces values from 46.0 to 48.3 μm with the same deviation of 0.6 μm. It can therefore be concluded from this data that the mean particle size of (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid) contained within xanthan wafers does not vary with statistical significance over a period of twelve weeks storage at either 25 or 40° C. This is clearly not the case for the gel suspension which shows a significant change in mean particle size from 47.5 to 62.0 μm with a larger average deviation of 1.8 μm over the same period of time.

Reference to FIG. 1 highlights the lack of variation between the particle size distributions at time zero (0 weeks) of non-irradiated wafers and wafers irradiated at 25 and 40 kGy respectively. Variations in the overall distribution are small and related to variations in sample concentration. In contrast, reference to FIG. 2 shows significant differences in distributions for gel suspensions at 0, 6 and 12 weeks storage at the higher temperature of 40° C. In particular an indisputable shift in peak values is clearly manifest from time zero, through six weeks to twelve weeks storage. Changes in particle size distribution between the first and last time points showing similar peak abundances of approximately 9% but a difference of 14.5 μm in the calculated mean values.

To fully appreciate the differences in the particle size distributions of wafers and gel suspensions, FIG. 3 consists of superimpositions of individual measurements for a twelve-week old non-irradiated wafer and the mother suspension stored at 40° C. Six separate measurements for both samples are made at two-minute intervals over a period of ten minutes and the differences in all values for the distributions are evident.

From the results of thermal analysis displayed in FIG. 4, it was clear that Lutrol F68 was present as a crystalline solid in the drug-loaded wafers as produced. On the first heating cycle, melting of this surfactant was evident around 50° C. in all three samples tested. This result alone indicated that no significant conversion of drug to the solvate (or ‘surfactantate’) had occurred during the period of accelerated ageing at 40° C. Furthermore, the absence of a Lutrol F68 melt on the second heating cycle provided proof of the reactivity of the contained drug substance with molten surfactant during the measurement.

4.6 Conclusions

It is apparent from the results described that hydrogel suspensions of insoluble drug are not stable of particle size. Although it is not possible from these measurements to discern a specific mechanism for the increases in particle size, observations and analysis of less stable gel suspensions (not discussed) suggest that aggregation is certainly involved. However, it can be stated with certainty that insoluble drugs contained within freeze-dried wafers do not appear to change their size after a period of accelerated ageing or after exposure to gamma-rays.

Thermal analysis served to confirm the instability of (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid) towards a fluid and reactive Lutrol F68 in support of knowledge relating to the formation of surfactantate in aqueous suspension. However, the inability of surfactantate to form in a solid, wafer formulation stored at 40° C. for three weeks provides further evidence of the stabilising properties of lyophilised wafers.

With respect to gamma-irradiation of drug-loaded wafers, there was no conclusive evidence from the DSC results that irradiated and non-irradiated wafers were thermally different. Identical results for all three samples, irrespective of radiation dose, suggested that the bulk form of the wafer and contained drug remained unchanged. In conclusion, these results are clear proof that wafers possess stabilising properties that hydrogel suspensions do not.

Other Formulations

The wound healing agent, compound A, is suspended in deionised water with the aid of a surfactant. Typically, 0.2% w/v Lutrol F-68 is used. Sodium carboxymethylcellulose (Blanose 7HF) is dissolved in the drug suspension at a concentration of 0.5-1.0% w/v and mixed until a uniform suspension is obtained. The mixture is poured in a suitable mould in a quantity commensurate with the desired size and shape of the final product. The drug loading may be varied from 0.1-60.0 mg/ml of the pre-lyophilised mixture, dependent on the desired dose per unit area. The cast mixture of polymer, surfactant and drug is then subjected to one of the preceding freeze-drying processes.

The above can be repeated with:

Sodium alginate at a concentration of 1-5% depending on the viscosity/molecular weight. For example,

sodium alginate, high viscosity, 2% solution (25° C.)=14.00 Pa.s

sodium alginate, med viscosity, 2% solution (25° C.)=3.50 Pa.s

sodium alginate, low viscosity, 2% solution (25° C.)=0.25 Pa.s

The wound healing agent, compound A, is suspended in deionised water with the aid of a surfactant. Typically, 0.2% w/v Lutrol F-68 is used. Low viscosity sodium alginate is dissolved in the drug suspension at a concentration of 5% w/v and mixed until a uniform suspension is obtained. The mixture is poured in a suitable mould and treated according to the previous examples.

1. A wafer composition comprising:
(a) a polymer substrate;
(b) a surfactant;
(c) water;
characterised in that the wafer further comprises stable, i.e. stable of size and form, crystalline particles of a pharmaceutically active wound healing agent.

2. A wafer according to claim 1 where the polymer substrate is xanthan gum.

3. A wafer according to either claim 1 or 2 where the surfactant is Lutrol F-68.

4. A wafer according to any one of claims 1 to 3 where the pharmaceutically active wound healing agent has a solubility in water in the range 1 ng/ml to 10 mg/ml.

5. A wafer according to any one of claims 1 to 4 where the pharmaceutically active wound healing agent is an MMP-3 and/or -13 inhibitor.

6. A wafer according to claim 5 where the MMP-3 and/or -13 inhibitor is (3R)-3-({[(1S)-2,2-Dimethyl-1-({[(1S)-2-methoxy-1-phenylethyl]amino}carbonyl)-propyl]amino}carbonyl)-6-[(3-methyl-4-phenyl)phenyl]hexanoic acid) or N-Hydroxy-4-[(4-{4-[6-(2-hydroxyethoxy)-2-pyridyl]-3-methylphenyl}-1-piperidinyl)sulfonyl]tetrahydro-2H-pyran-4-carboxamide).

7. A wafer according to any one of claims 1 to 5 where the pharmaceutically active wound healing agent is a UPA inhibitor.

8. A wafer according to claim 7 where the UPA inhibitor is 3-(4-Chloro-1-guanidino-7-isoquinolyl)benzoic acid) or 2-[(4-Chloro-1 -guanidino-7-isoquinolinyl)sulfonylamino]isobutyric acid).

9. A wafer according to any one of claims 1 to 8 where the concentration of the wound healing agent is 1 to 60 mg/ml of the pre-formed mixture.

10. A wafer according to any one of claims 1 to 9, which is lyophilised.

11. Use of a wafer according to any one of claims 1 to 10 in wound healing.

12. A process for the preparation of a lyophilised wafer according to any one of claims 1-10 which comprises the steps of:
(a) forming a suspension by mixing a polymer substrate, a surfactant and a wound healing agent in water;
(b) shaping the suspension into a suitable mould; and
(c) freeze drying the shaped composition.

13. A process according to claim 12, which additionally includes the step:
(d) sterilising the resulting wafer.

14. A process according to claim 13 or 14 where the polymer substrate is xanthan and it is dissolved in a concentration of about 0.5% w/v.
